(19) United States
(12) Patent Application Publication
(10) Pub. No.: US 2015/0282978 A1
(43) Pub. Date: Oct. 8, 2015

(54) Low-Modulus, Halogen-Free, Odor Containment Barrier

(71) Applicant: AVERY DENNISON CORPORATION, Pasadena, CA (US)

(72) Inventor: Kevin O. Henderson, Willoughby Hills, OH (US)

(21) Appl. No.: 14/371,618

(22) PCT Filed: Jan. 9, 2013
(86) PCT No.: PCT/US2013/020721

§ 371 Date: Jul. 10, 2014

Related U.S. Application Data
(60) Provisional application No. 61/584,471, filed on Jan. 9, 2012.

(51) Int. Cl. A61F 5/441; A61L 31/06; A61L 31/14; A61F 5/448; A61L 31/04

(52) U.S. Cl. CPC A61F 5/441; A61F 5/448; A61L 31/048; A61L 31/14; A61L 31/06

(57) ABSTRACT

A multilayer barrier assembly having excellent barrier properties, low modulus, and which is halogen free, is described. The barrier assembly can be used in numerous medical applications and particularly in films for ostomy products. In one version of the barrier assembly, a layer is used that includes a variant form of ethylene vinyl alcohol. In another version of the barrier assembly, a layer is utilized that includes one or more elastomeric norbornenes. The barrier assemblies may also utilize combinations of these layers.

### CROSS REFERENCES TO RELATED APPLICATIONS

The present application claims the benefit of U.S. Provisional Patent Application No. 61/584,471 filed Jan. 9, 2012, which is incorporated herein by reference in its entirety.

### FIELD

The present subject matter relates to compositions, films, and multilayer barrier assemblies for use in medical applications and particularly for use in ostomy films.

### BACKGROUND

Barrier films in medical applications and particularly as used in ostomy applications, typically contain halogens. An example of a material used in such applications is polyvinylidene chloride (PVDC). Although use of that material is satisfactory in many regards, films containing halogens such as chloride and bromide are difficult and costly to recycle. In fact, with increasing environmental awareness, many regulations prohibit the disposal of halogens, thereby further increasing the inconvenience and/or cost of handling used medical products containing halogens.

Prior artisans have investigated the use of other agents or materials in place of halogens. One such agent is ethylene vinyl alcohol (EVOH). While ethylene vinyl alcohol generally provides good barrier properties, it is not suitable for all applications. When utilizing lower ethylene content EVOH, which is required for higher barrier levels, the resulting material is very sensitive to moisture. And, the material also typically exhibits a relatively high modulus. When used in thin film applications such as ostomy barrier films, a high modulus is undesirable due to the relatively high levels of noise that the films generate when distorted or flexed.

Accordingly, a need exists for a barrier material that can be used in various medical applications, and particularly in ostomy applications. The material should exhibit good barrier properties, be halogen-free, and exhibit a relatively low modulus so that films formed from such materials, are quiet and do not generate excessive amounts of noise.

### SUMMARY

The difficulties and drawbacks associated with previously known materials, films and related products are addressed in the present subject matter for a unique multilayer barrier assembly.

In one aspect, the subject matter provides a multilayer barrier assembly comprising a first layer including at least one of EVOH and G-EVOH, and a second layer including one or more elastomeric norbornenes. The first and second layers when combined, exhibit a 2% secant modulus of less than 30 kpsi in both a machine direction (MD) and a cross direction (CD).

In another aspect, the subject matter provides a multilayer barrier assembly comprising at least one layer including G-EVOH.

In still another aspect, the subject matter provides a multilayer barrier assembly comprising an outer layer; a first flexible support; a first tie layer including a tie component; a barrier layer including at least one of EVOH, G-EVOH, PGA material, and metal oxide(s); a second tie layer including a tie component; a second flexible support; and an inner layer.

In yet another aspect, the present subject matter provides an ostomy barrier assembly comprising an outer layer; a first flexible support; a first tie layer including a tie component; a barrier layer including at least one of EVOH, G-EVOH, PGA material, and metal oxide(s); a second tie layer including a tie component; a second flexible support; and an inner layer.

In another aspect, the subject matter provides a multilayer barrier assembly comprising an outer layer, a first flexible support, a second flexible support, a first general barrier layer, a barrier layer including EVOH, a second general barrier layer, a third flexible support, a functional layer, and an inner layer.

As will be realized, the subject matter is capable of other and different embodiments and its several details are capable of modifications in various respects, all without departing from the subject matter. Accordingly, the drawings and description are to be regarded as illustrative and not restrictive.

### BRIEF DESCRIPTION OF THE DRAWINGS

FIG. 1 is an exploded schematic view of a preferred embodiment multilayer barrier in accordance with the present subject matter.

FIG. 2 is an exploded view of another preferred embodiment multilayer barrier in accordance with the present subject matter.

FIG. 3 is an exploded view of another preferred embodiment multilayer barrier in accordance with the present subject matter.

### DETAILED DESCRIPTION OF THE EMBODIMENTS

The present subject matter provides new barrier films and multilayer barrier assemblies using such films that uniquely provide excellent barrier properties, low modulus and thus low noise, and characteristics that render the barrier assemblies amenable to recycling. Preferably, in certain embodiments, the multilayer barrier assemblies comprise at least one and most preferably a combination of (i) a film comprising a variant of ethylene vinyl alcohol, and (ii) a film comprising one or more elastomeric norbornenes. The preferred barrier assemblies exhibit properties that are comparable to films containing a high ethylene-content EVOH, while maintaining the functionality of a low ethylene content EVOH. Preferably, in certain additional embodiments, the multilayer barrier assemblies comprise at least one and most preferably a combination of (i) a film comprising ethylene vinyl alcohol, and (ii) a film comprising one or more norbornenes such that the films when combined such as by coextruding or laminating, exhibit a 2% secant modulus of less than 30 kpsi in both a machine direction (MD) and in a cross direction (CD). The preferred barrier assemblies also exhibit excellent barrier properties to odor molecules, while still featuring low noise characteristics.

### Ethylene Vinyl Alcohol

Ethylene vinyl alcohol, commonly abbreviated EVOH, is a formal copolymer of ethylene and vinyl alcohol. Because the latter monomer mainly exists as its tautomer acetaldehyde, the copolymer is prepared by polymerization of ethylene and vinyl acetate to produce the ethylene vinyl acetate (EVA) copolymer followed by hydrolysis. EVOH is typically used to provide barrier properties, primarily as an oxygen barrier for improved food packaging shelf life and as a hydrocarbon barrier for fuel tanks. EVOH is typically coextruded or laminated as a thin layer between cardboard, foil, or other plastics. EVOH copolymer is traditionally defined by the mole percent ethylene content. Lower ethylene content grades have higher barrier properties; and higher ethylene content grades have lower melting temperatures for ease in extrusion.

As explained herein, the preferred embodiment multilayer barrier assemblies preferably include a film which comprises a variant of EVOH. These variant EVOH polymers exhibit relatively low crystallinity and low melting point, and also exhibit high transparency, orientability, and shrinkability. Several preferred variants of EVOH are commercially available under the designation G-EVOH from Nippon Gohsei of Osaka, Japan. Details as to EVOH variants for incorporation in one or more layers of the preferred multilayer barrier assemblies are described in greater detail herein.

In certain embodiments, it is preferred to use a particular grade of EVOH which contains approximately 44 mol % ethylene and which has been modified for thermoforming. This grade of EVOH exhibits higher barrier properties than 48 mol % ethylene grades, but also exhibits a lower modulus than a standard 44 mol % ethylene grade. A preferred type of this grade of EVOH is commercially available from Kuraray or Eval Company of America of Houston, Tex. under the designation EVAL SP292. This material is described in greater detail herein.

### Elastomeric Norbornenes

Norbornene is a bicyclic olefin. Norbornene is a bridged six-membered ring with a double bond on one side. The bridged ring places extra strain on the double bond, making it highly reactive. Therefore, many opportunities exist to modify the basic norbornene molecule or to incorporate that molecule into larger molecules, such as cyclic olefin copolymer (COC) or other polymers. The processes used for such modifications may include ring-opening metathesis polymerization (ROMP), radical or cationic polymerization, and vinyl or addition polymerization.

Norbornene is typically produced by the Diels-Alder reaction of cyclopentadiene and ethylene, two commonly available hydrocarbons. Norbornene is a colorless substance with a melting point of 47° C. (115° F.), and is soluble in many common solvents. As well as being used in various COC materials such as those commercially available from Topas Advanced Polymers, norbornene has numerous other applications including use in pharmaceutical intermediates, pesticide compounds, specialty fragrances and high-damping rubber. Norbornene has also been used in organic synthesis and because of its combination of strong odor and low toxicity, it is also used as an odorant.

The various films and barrier assemblies as described herein utilize an elastomeric norbornene. The preferred elastomeric norbornene material is an elastomeric form of cyclic olefin copolymer (COC). A preferred elastomeric norbornene is commercially available from TOPAS under the designation E-140. Details as to the elastomeric norbornene for incorporation in one or more layers of the preferred multilayer barrier assemblies are described in greater detail herein.

In certain embodiments, it is preferred to combine effective amounts of one or more elastomeric COCs and EVOH in a layer of a multilayer barrier assembly. Surprisingly, it has been found that a multilayer film containing effective amounts of COCs and EVOH is sufficiently flexible yet exhibits excellent barrier properties. Preferably, the film comprising one or more elastomeric COCs and EVOH which exhibits a 2% secant modulus less than 30 kpsi in a machine direction (MD) and a cross direction (CD). Table 1 set forth below lists 2% secant modulus values for films having (i) EVOH, (ii) EVOH and rigid COC, (iii) an elastomeric COC, and (iv) EVOH and elastomeric COC.

TABLE 1
Flexibility and Odor Blocking Characteristics
for Films with EVOH and/or COC

| | 2% Secant | | |
|---|---|---|---|
| | Modulus (kpsi) | | Odor Rating |
| | MD | CD | (5 = bad) |
| Cast Film with EVOH | 30.4 | 10.5 | 3.5 |
| Cast Film with EVOH and Rigid COC | 65.9 | 64.9 | 1.0 |
| Cast Film with Elastic COC | 1.8 | 1.1 | 5.0 |
| Cast Film with EVOH and Elastic COC | 23.1 | 16.4 | 2.0 |

### Multilayer Barrier Assembly

The present subject matter also provides multilayer barrier assemblies for use in medical applications and in particular, for ostomy applications. As will be appreciated, an important characteristic for such barrier assemblies is preventing or at least significantly reducing transmission of odors through the barrier. Another important characteristic for such barriers is that the barriers be relatively quiet and not emit excessive noise upon deflecting or movement of the layered assembly. Several preferred barrier assemblies feature at least one of (i) a polymeric barrier layer comprising G-EVOH and (ii) a barrier layer comprising one or more elastomic norbornenes. A most preferred multilayer barrier construction uses one or more barrier layers that include G-EVOH and one or more barrier layers including elastomeric norbornenes.

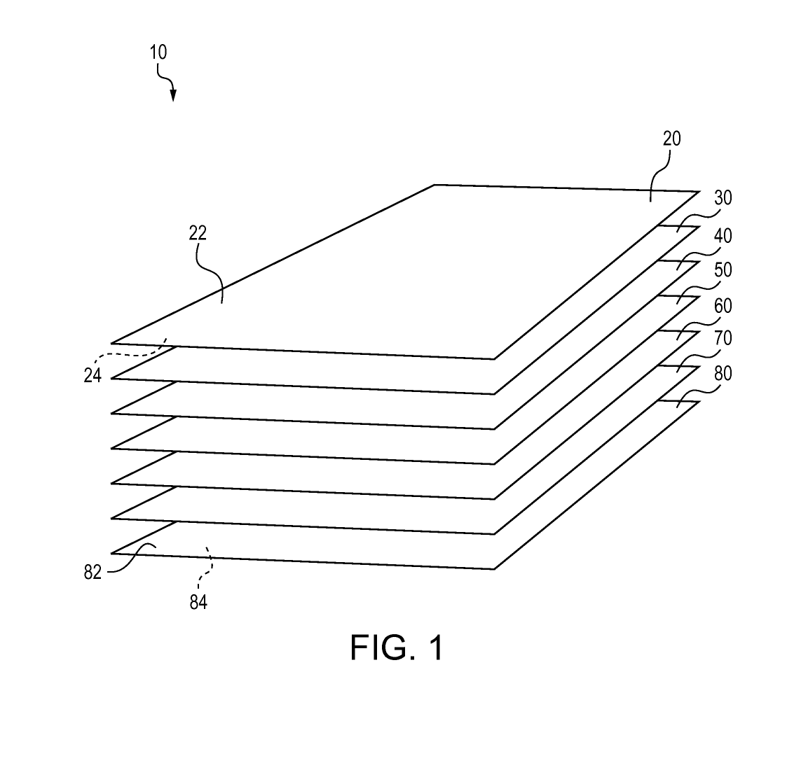

FIG. 1 is a schematic exploded illustration of a preferred multilayer barrier assembly 10 in accordance with the present subject matter. The multilayer assembly 10 comprises an outer layer 20 defining an outer face 22 and an oppositely directed inner face 24, a first flexible support layer 30, a first moisture and odor barrier layer(s) 40, a secondary barrier 50 for reducing transmission of oxygen and odors, a second moisture and odor barrier 60, a second flexible support layer 70, and an inner layer 80 defining an inner face 84. Optionally, this inner layer contains an antimicrobial layer or agent for contacting a microbe containing medium. The layer 80 also defines an oppositely directed face 82.

More specifically, referring to Table 2, the preferred embodiment multilayer barrier assembly 10 includes (the layers are noted in order from the outermost layer to the innermost layer): an outer nonwoven layer 20; a first flexible support 30 for dampening noise, preferably using an elastomer of polyethylene and polypropylene; a first moisture and odor barrier layer 40 preferably including the elastomeric COC and a compatibilizer, g-maleic anhydride ethylene vinyl acetate (GMAH-EVA); another barrier 50 for reducing transmission of oxygen and hydrogen sulfide (i.e. odor) that includes EVOH; a second moisture and odor barrier 60 corresponding to previously noted layer 40; a second flexible support 70 corresponding to previously noted layer 30; and an inner layer 80. Preferably, the layer(s) 20-80 are arranged in order and each layer is disposed immediately adjacent to one or more sequentially positioned layer(s). For example, layer 30 is preferably disposed immediately adjacent and between layers 20 and 40. However, it will be appreciated that the subject matter includes other multilayer assemblies using different arrangements and combinations of layers.

TABLE 2
Preferred Multilayer Barrier

| | Weight | | MFR (dg/min) | |
|---|---|---|---|---|
| Layer | Percent | Layer Description | 190° C. | 230° C. |
| 20 | 10% | 70% EVA (18% VA) | 3.00 | |
| | | 15% Metallocene-Catalyzed LLDPE | 5.50 | |
| | | 15% Slip/Antiblock | | |
| 30 | 28% | Polyolefin Elastomer | | 7.00 |
| 40 | 7% | 75% Elastomeric COC | 2.82 | |
| | | 25% GMAH-Masterbatch | | |
| 50 | 10% | 100% 48 mol EVOH | 6.90 | 15.00 |
| 60 | 7% | 75% Elastomeric COC | 2.82 | |
| | | 25% GMAH-Masterbatch | | |
| 70 | 28% | Polyolefin Elastomer | | 7.00 |
| 80 | 10% | 85% EVA (12% VA) | 3.00 | |
| | | 15% Slip/Antiblock | | |

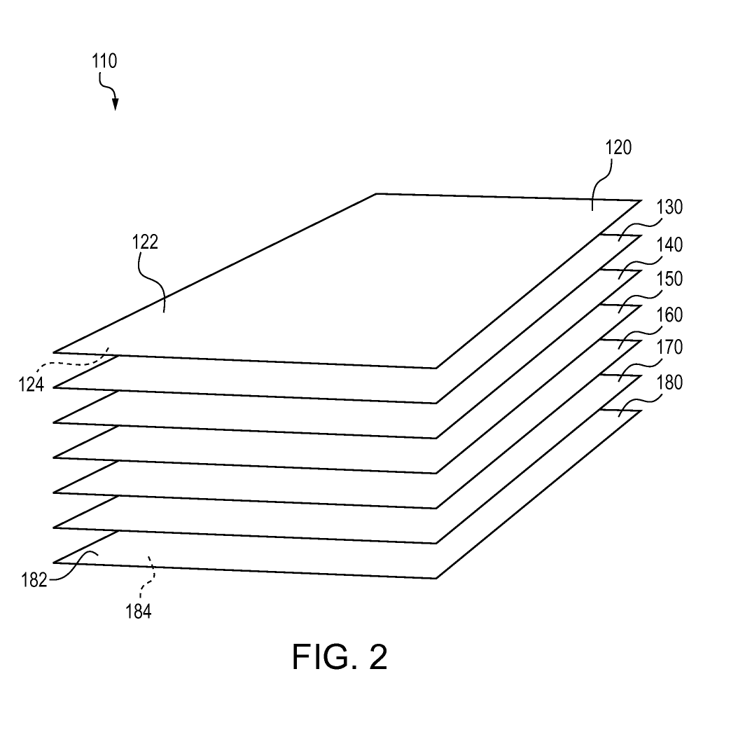

FIG. 2 is a schematic exploded illustration of another preferred multilayer barrier assembly 110 in accordance with the subject matter. The multilayer assembly 110 comprises an outer layer 120 defining an outer face 122 and an oppositely directed inner face 124, a first flexible support layer 130, a tie layer 140, a moisture and odor barrier layer 150, a second tie layer 160, a second flexible support layer 170, and an inner barrier layer 180. The inner layer 180 defines an inner face 184 and an oppositely directed face 182.

More specifically, referring to Table 3, the preferred embodiment multilayer barrier assembly 110 is further described. That barrier 110 includes an outer nonwoven layer 120; a first flexible support 130 for dampening noise, preferably using an elastomer of polyethylene and polypropylene, a first tie layer 140; a moisture and odor barrier 150 for reducing transmission of oxygen and odor such as hydrogen sulfide preferably using G-EVOH; a second tie layer 160; a second flexible support 170 as previously described with regard to layer 130; and an inner layer 180 that serves as a barrier layer. The inner layer 180 preferably includes an elastomeric COC.

TABLE 3
Preferred Multilayer Barrier

| | Weight | | MFR (dg/min) | |
|---|---|---|---|---|
| Layer | Percent | Layer Description | 190° C. | 230° C. |
| 120 | 10% | 70% EVA (18% VA) | 3.00 | |
| | | 15% Metallocene-Catalyzed LLDPE | 5.50 | |
| | | 15% Slip/Antiblock | | |
| 130 | 30% | 100% Polyolefin Elastomer | | 3.00 |
| 140 | 5% | 100% GMAH-EVA | 10.90 | |
| 150 | 8% | 100% 38 mol G-EVOH (processes | | 3.50 |
| | | like 48 mol EVOH) | | |
| 160 | 5% | 100% GMAH-EVA | 10.90 | |
| 170 | 30% | 100% Polyolefin Elastomer | | 3.00 |
| 180 | 12% | 85% Elastomeric COC | 2.82 | |
| | | 15% Slip/Antiblock | | |

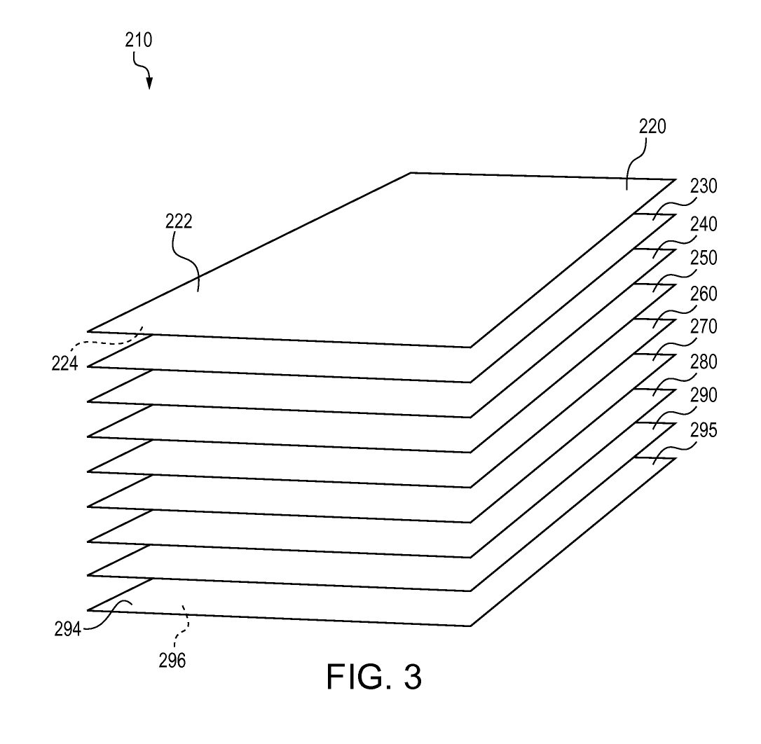

FIG. 3 is a schematic exploded illustration of another preferred multilayer barrier assembly 210 in accordance with the subject matter. The multilayer assembly comprises an outer layer 220 defining an outer face 222 and an oppositely directed inner face 224, a first flexible support layer 230, a second flexible support layer 240, a first general barrier layer 250, a moisture and odor barrier 260 for reducing transmission of oxygen and odor such as hydrogen sulfide, a second general barrier layer 270, a third flexible support layer 280, a functional layer 290, and an inner layer 295 defining an inner face 294 and an oppositely directed face 296.

More specifically, referring to Table 4, the preferred embodiment multilayer barrier assembly 210 is further described. That barrier 210 includes an outer layer 220 comprising ethylene vinyl acetate and one or more slip and antiblock agents. The barrier 210 also includes the support layer 230 which may preferably include one of two blends of materials described in greater detail below. The barrier 210 also includes support layers 240 and 280 which preferably include one of two particular materials. The barrier 210 further includes layers 250 and 270 which utilize one or more elastomeric COC's. The barrier 210 also includes the moisture and odor barrier 260 which preferably includes a particular grade of ethylene vinyl alcohol, i.e. SP292. The functional layer 290 preferably includes ethylene butyl acrylate (EBA) preferably utilized in combination with one or more other components. And, the barrier 210 also includes an inner layer 295 that preferably includes one or more antimicrobial agents.

TABLE 4
Preferred Multilayer Barrier

| | | | Layer |
|---|---|---|---|
| | | | Thick- |
| Layer | Description | | ness |
| 220 | 80% EVA (18% VA) | | 10% |
| | 20% slip and antiblock masterbatch | | |
| 230 | 80% NOTIO ™ | 80% AFFINITY ™ EG 8100G | 14% |
| | 20% VISTAMAXX ™ | 20% Tie | |
| 240 | NOTIO ™ | AFFINITY ™ EG 8100G | 14% |
| 250 | 80% Elastomeric COC | | 8% |
| | 20% Tie | | |
| 260 | EVOH SP292 | | 7% |
| 270 | 80% Elastomeric COC | | 8% |
| | 20% Tie | | |
| 280 | NOTIO ™ | AFFINITY ™ EG 8100G | 9% |
| 290 | 60% EBA 30 | 60% EBA 30 | 20% |
| | 20% NOTIO ™ | 20% AFFINITY ™ EG 8100G | |
| | 20% Tie | 20% Tie | |

| | | |
|---|---|---|
| 295 | 70% EBA 30 | 10% |
| | 20% Slip and antiblock masterbatch | |
| | 10% Silver ion anti-microbial masterbatch | |

Layer 220 is the outermost layer and comprises ethylene vinyl acetate (EVA) having a vinyl acetate content of 18%. Layer 220 will also contain a masterbatch of slip and anti block. The masterbatch is SAB1220 NG masterbatch (A. Schulmann product) added at 20% by weight. The masterbatch contains 8% slip and 12% antiblock agents. The slip agent is believed to be erucamide and the antiblock agent is believed to be SiO₂. Optionally, an effective amount of a tie-component can be used in this layer. If a tie component is used in layer A, the material is BYNEL E418, available from DuPont. That material is described as anhydride-modified EVA, and is believed to be maleic anhydride grafted EVA polymer.

Layer 230 is one of two compositions. Either a blend of 80% NOTIO™ and 20% VISTAMAXX™ is used or a blend of 80% AFFINITY™ EG 8100G and 20% of a tie component is used. NOTIO™ is a nano-crystal structure controlled elastomer available from Mitsui Chemicals. The Notio material is NOTIO PN2070. This is an elastomeric material and does not contain nanoclays. VISTAMAXX is propylene based elastomers available from Exxon Mobil Chemical. A preferred material for the tie component is an adhesive resin available from DuPont under the designation BYNEL. The tie component is preferably BYNEL CXA410E710, which is believed to be an anhydride modified olefin.

Layers 240 and 280 each include either NOTIO™ or AFFINITY™ EG 8100G.

Each of layers 250 and 270 is 80% elastomeric cyclic olefin copolymer (COC) and 20% tie component. The proposed COC is from TOPAS under the designation E-140 and has a density of 0.94 g/cc and a comonomer content of about 12% Norbornene. The E-140 COC is a semi-crystalline material and has a melting point of about 84° C. and a glass transition temperature Tg of about 0° C. It is also contemplated that two grades of COC from TOPAS, grade 9506 and/or grade 8007, may be blended with the E-140 grade at a blend ratio of 50/50 by weight). In Layers 250 and 270, the tie component is BYNEL CXA410E710. It is believed that this is an anhydride modified olefin.

Layer 260 is ethylene vinyl alcohol (EVOH) SP292 available from Eval Americas.

Layer 290 is either a blend of 60% ethylene butyl acrylate (EBA) 30, 20% NOTIO™ or AFFINITY™ EG 8100G, and 20% of a tie component. The tie component is preferably BYNEL E418, which is believed to be maleic anhydride grafted EVA.

Layer 295 is 70% EBA 30, 20% of a slip and antiblock masterbatch, and 10% of a silver ion anti-microbial masterbatch. In layer 295, 20% by weight of the SAB1220 NG masterbatch is used. In layer 295, the anti-microbial masterbatch is ABACT 421P from A. Schulman. However, it is also contemplated that such anti-microbial masterbatch may be substituted with BACTIBLOCK from Nanobiomatters. The Nanobiomatter anticrobial is a silver ion on a modified organoclay.

The previously noted Table 4 includes the expected thickness in percentage of each layer based upon total thickness of the multilayer film: 10/14/14/8/7/8/9/20/10. Other slight variations in thickness are contemplated. The total thickness of the three middle layers is about 30% or less of the total thickness of the multilayer film. Film thickness for an ostomy application is about 70-100 microns, but could be thinner or thicker.

The proposed film construction is believed to exhibit several advantages over currently known ostomy films. The proposed film is halogen-free and avoids the use of polyvinylidene chloride (PVDC). The proposed film is quieter and exhibits significantly less “rustle”. And, the proposed film appears to exhibit superior odor blocking characteristics. The film construction may be transparent or contain coloring agents

In certain embodiments, it is preferred that one or more layers including at least one COC is provided between an innermost layer of the multilayer barrier assembly and the layer(s) containing EVOH or G-EVOH. Referring to Table 5, it can be seen that if the layer containing COC is located toward the exterior of the multilayer barrier assembly, odor blocking ability is significantly and detrimentally impacted.

TABLE 5
Effect of Location of COC Layer

| | Outer | | | | Inner | Odor |
|---|---|---|---|---|---|---|
| | Layer | | | | Layer | Rating |
| Layer % | 16.8% | 34.1% | 5.0% | 34.1% | 10.0% | (5 = Bad) |
| Asym 1 | 60% | Elastic | EVOH | Elastic | EVA/ | 5.0 |
| | COC | PO | | PO | AM | |
| Asym 1 | 24% | Elastic | EVOH | Elastic | EVA/ | 5.0 |
| | COC | PO | | PO | AM | |

It has also been surprisingly discovered that a continuous EVOH layer is preferred over thinner separate layers containing EVOH. When welded blown films (Table 6) were compared to standard films (Table 7), the standard films performed better in regards to blocking odor as compared to the welded films. If the layer thickness is consistent and the amount of elastomeric COC in the blend is reduced, odor containment begins to decrease.

TABLE 6
Odor Blocking Characteristics of Welded Multilayer Barriers

| | | | | 80% | | | | 80% | | | |
|---|---|---|---|---|---|---|---|---|---|---|---|
| | | | | COC | | | | COC | | | |
| | | Outer | | + | | Elastic | | + | | Inner | Odor |
| | | Layer | Elastic | 20% | | PO + | | 20% | | Layer | Rating |
| | Component | EVA/AM | PO | tie | EVOH | tie | EVOH | tie | Elastic | EVA/AM | (5 = bad) |
| Weld 1 | Layer % | 8.5% | 19.0% | 6.0% | 2.5% | 28.0% | 2.5% | 6.0% | PO | 8.5% | 2.5 |
| Weld 2 | Layer % | 8.5% | 20.5% | 4.50% | 2.5% | 28.0% | 2.5% | 4.5% | 20.5% | 8.5% | 2.0 |
| Weld 3 | Layer % | 8.5% | 22.5% | 2.5% | 2.5% | 28.0% | 2.5% | 2.5% | 22.5% | 8.5% | 1.5 |

TABLE 7
Odor Blocking Characteristics of Standard Multilayer Barriers

| | Outer | | | | | | Inner | Odor |
|---|---|---|---|---|---|---|---|---|
| | Layer | | | | | | Layer | Rating |
| Layer % | 16.8% | 24.7% | 6.0% | 5.0% | 6.0% | 24.7% | 16.8% | (5 = bad) |
| Sym 1 | EVA | Elastic PO | 80% COC | EVOH | 80% COC | Elastic PO | EVA/AM | 1.0 |
| Sym 2 | EVA | Elastic PO | 60% COC | EVOH | 60% COC | Elastic PO | EVA/AM | 1.0 |
| Sym 3 | EVA | Elastic PO | 35% COC | EVOH | 35% COC | Elastic PO | EVA/AM | 1.5 |

Based upon the foregoing and other aspects of the preferred embodiment multilayer barrier films or film assemblies, generally a preferred thickness for the layer containing elastomeric COC and which is located between the innermost layer and the layer(s) containing EVOH or G-EVOH, is at least 0.18 mils. Preferably, the content of elastomeric norbornene in the COC-containing layer is at least 50% by weight.

Referring further to Tables 2, 3, and 4, the outer nonwoven layer, i.e. 20, 120, and 220, preferably includes a sublayer indicated by the reference to 18% vinyl acetate (VA). This sublayer is provided for receiving attachments to the multilayer film. Melt flow rates (MFR) are given at two different temperatures for the various layers. One or more noise suppressants may optionally be included in at least one of the layers for moisture and odor barrier control and the layer for reducing transmission of oxygen and odors such as hydrogen sulfide. The percentages shown in Tables 2 and 3 do not include addition of the noise suppressant(s).

The COC preferred for use in the multilayer barrier constructions is an elastomeric COC. The COC can be used in a layer in nearly any concentration, such as from about 10% to about 100%, more preferably from about 14% to about 100%, more preferably from about 50% to about 90%, more preferably from about 60% to about 80%, and most preferably from about 75% to about 85%, based upon the weight of the layer. Although not wishing to be bound to any particular proportions, when using an elastomeric COC, it is preferred that the layer comprising the elastomeric COC constitute from about 5% to about 15% of the total weight of the multilayer barrier. In certain embodiments, it is preferred to use the elastomeric COC in combination with a tie component such as g-maleic anhydride ethylene vinyl acetate (GMAH-EVA). The multilayer barrier 10 represents such a combination. In other embodiments, the elastomeric COC is used in combination with a slip agent and/or an antiblock agent. The multilayer barrier 110 is an example of such a combination. In still other embodiments, the elastomeric COC is used in combination with one or more tie components and/or incorporated in two or more layers of the multilayer barrier assembly. The multilayer barrier 210 is an example of such features. It will be appreciated that the various multilayer barrier films are not limited to the use of these particular elastomeric COC's. Instead, it is contemplated that a wide range of comparable compounds and/or materials could be utilized in the noted moisture and odor barrier layer(s) of the multilayer construction. As previously noted, in certain embodiments, it may be preferred to include one or more noise suppressants in the layer(s) containing the COC's.

The barrier for reducing transmission of oxygen and odor such as hydrogen sulfide, preferably comprises EVOH and most preferably G-EVOH. That is, the preferred multilayer barrier assemblies preferably include a layer that includes at least one of EVOH and G-EVOH. The multilayer barrier 10 illustrates use of a layer including EVOH. And, the multilayer barrier 110 is an example of a layer including G-EVOH. The EVOH or G-EVOH is incorporated at nearly any effective concentration, however typical concentrations in a layer range from about 40% to about 100%, preferably from about 50% to about 100%, and most preferably from about 60% to about 100%. For certain applications, it is contemplated that G-EVOH be used and preferably a particular grade of ethylene vinyl alcohol copolymer commercially available under the designation G-SOARNOL from Nippon Gohsei can be used. As compared to conventional EVOH, G-SOARNOL polymers exhibit relatively low crystallinity and low melting point, and relatively high transparency, orientability, and shrinkability. The G-SOARNOL polymer system exhibits increased barrier properties as compared to conventional EVOH. Although not wishing to be bound to any particular theory, it is believed that G-SOARNOL is commercially available from Nippon under the designations SG634B and SG654B. These materials are believed to be copolymers of EVOH and polyvinyl alcohol (PVA). The SG654B material is reported to exhibit a melt flow rate of 3.5 g/10 min (ISOI 130, 230° C., 2.16 kg). The use of G-SOARNOL imparts to the resulting layer a higher gas barrier at a lower modulus. Essentially, the G-SOARNOL performs as a high ethylene content EVOH while maintaining the functionality associated with a low ethylene content EVOH. G-EVOH is also known as G-Polymer.

In certain embodiments and particularly for the preferred multilayer barrier in Table 4, it is preferred to utilize a grade of EVOH which contains 44 mol % ethylene and which has been modified for thermoforming. A preferred type of this grade of EVOH is commercially available from Kuraray or Eval Company under the designation EVAL SP292. Physical properties of this preferred grade of EVOH are set forth below in Table 8:

TABLE 8
Physical Properties of EVAL SP292

| | | |
|---|---|---|
| Unit | Test Method | Value |

Standard Properties

| | | | |
|---|---|---|---|
| Melt Index - 190° C., 2160 g | g/10 min | ISO 1133 | 2.0 |
| 210° C., 2160 g | | | 4.5 |
| Density | g/cm³ | ISO 1183 | 1.13 |

Thermal Properties

| | | | |
|---|---|---|---|
| Melt Temperature | ° C. (° F.) | ISO 11357 | 161 (321) |
| Crystallization Temperature | ° C. (° F.) | ISO 11357 | 141 (285) |
| Glass Transition Temperature | ° C. (° F.) | ISO 11357 | 48 (118) |

Physical Properties

| | | | |
|---|---|---|---|
| Tensile Strength at Break | MPa (psi) | ISO 527 | 25 (3626) |
| Elongation at Break | % | ISO 527 | 17 |
| Young's Modulus | MPA (psi) | ISO 527 | 2300 (333, 592) |
| Limit of Orientability | Simultaneous | Kuraray Method | 4.5 × 4.5 |
| | Sequential | Kuraray Method | 4 × 4 |

Barrier Properties

| | | | |
|---|---|---|---|
| O₂ Transmission Rate | | | |
| 65% RH, 20° C. | cm³ · mil/100 in² · day · atm | ISO 14663-2 | 0.090 (1.8) |
| 85% RH, 20° C. | (cc · 20 μm/m² · day · atm) | | 0.215 (4.3) |

The outer layer in the preferred barrier assemblies, such as outer layers 20, 120, and 220 is preferably in the form of a nonwoven material comprising a majority proportion of ethylene vinyl acetate (EVA). The outer layer may also comprise a minority proportion of metallocene-catalyzed linear low density polyethylene (LLDPE). Various slip agents and/or antiblock agents can also be utilized in the outer layer.

The flexible supports in the preferred multilayer constructions such as layers 30, 70, 130, 170, 230, 240, and 280, utilize a low density polyolefin and preferably, a polyolefin elastomer. A wide array of commercially available polyolefin elastomers can be used for one or both of the flexible support layers. Representative preferred examples of such materials include KRATON™ D1164P and G2832 available from Kraton Polymers US, LLC of Houston, Tex.; DOW AFFINITY™ EG 8200 and DOW VERSIFY™ 3200 and 3000 from Dow Chemical Corp. of Midland, Mich.; DYNAFLEX™ G2755 from GLS Corp. of McHenry, Ill.; SEPTON™ 2063 from Kuraray of Tokyo, Japan; and VISTAMAXX™ VM1100 from ExxonMobil Chemical Co. of Houston, Tex. Table 9 set forth below presents representative modulus, tear strength, and density values for films made using these materials.

TABLE 9
Summary of Modulus, Tear Strength and Density

| | Modulus | | Tear Strength | | |
|---|---|---|---|---|---|
| | [MPa] | | [g] | | Density |
| Core Resin Name | MD | TD | MD | TD | [g/cm³] |
| KRATON ™ D1164P | 40.3 | 9.6 | 95 | 776 | 0.96 |
| KRATON ™ G2832 | 5.0 | 1.7 | 179 | 207 | 1.01 |
| DOW AFFINITY ™ EG 8200 | 8.8 | 8.1 | 150 | 188 | 0.93 |
| DYNAFLEX ™ G2755 | 9.8 | 3.2 | 175 | 186 | 0.89 |
| DOW VERSIFY ™ 3200 | 52.8 | 59.3 | 368 | 1026 | 0.93 |
| DOW VERSIFY ™ 3000 | 128.9 | 138.4 | 554 | 1205 | 0.87 |
| Kuraray SEPTON ™ 2063 | 8.0 | 8.1 | 83 | 56 | 1.00 |
| Exxon VISTAMAXX ™ | 7.1 | 7.6 | 108 | 111 | 0.98 |
| VM1100 | | | | | |

The tie layer if used, such as layers 140 and 160 in the preferred multilayer barrier assembly 110, or layers 250, 270, and 290 in the barrier 210, can include one or more tie agents. The tie layer serves to couple or secure adjacent layers together. For example, the tie layer 140 serves to affix the support layer 130 with the barrier layer 150. As previously noted, a preferred tie agent is GMAH-EVA. However, in no way is the subject matter or any of its embodiments limited to that particular agent.

The inner layer such as layers 80, 180, and 295 preferably comprises an antimicrobial agent. In certain embodiments, the antimicrobial layer also preferably comprises one or more sealable polymers such as metallocene-catalyzed linear low density polyethylene (LLDPE) and ethylene vinyl acetate (EVA). One or more slip and/or antiblocking agents can also be incorporated into this layer. It is also contemplated that one or more blowing agents could be incorporated into the inner layer.

### Additional Materials for Barrier Layer(s)

The present subject matter also includes the use of other materials in barrier layer(s) in addition to or instead of, the EVOH materials described herein. For example, in certain versions of the present subject matter, a barrier layer is provided which comprises an effective amount of polyglycolic acid (PGA) and/or PGA resin(s). The terms “PGA” or “PGA material” as used herein refer to certain polymeric materials produced or based upon glycolic acid. Typically, the PGA materials are thermoplastic polyesters and are biodegradable. The terms PGA or PGA material as used herein also include various PGA resins. In certain embodiments, the polyglycolic acid (PGA) resin generally includes one or more homopolymers of glycolic acid which include recurring units of —(—O—CH₂—CO—)— and one or more glycolic acid copolymers containing at least 50% by weight of the noted glycolic acid recurring unit. In particular embodiments, the content of the noted glycolic acid recurring unit in the PGA resin is at least about 50%, more particularly at least 70%, and more particularly at least 90% by weight. The PGA resin typically has a weight average molecular weight (based on polymethyl methacrylate) in a range of 30,000 to 800,000 according to GPC measurement using hexafluoroisopropanol solvent. In certain embodiments, the weight average molecular weight is in a range of 150,000 to 300,000. More particularly and in certain embodiments, a semicrystalline polyester polyglycolic acid resin can be used. An example of such a resin which is useful has a specific gravity of 1.50 g/cm³ in the amorphous state and 1.70 g/cm³ in the crystalline state with a maximum degree of crystallinity of about 50%. Such PGA resins are available from Kureha America, Inc. under the designation KUREDUX™. These resins are biodegradable, exhibit relatively high moduli, and are very hydroscopic. When incorporated in a barrier film or layer, the thickness of the barrier layer could, if desired, be relatively thin. In certain versions of the present subject matter, the thickness could be as low as approximately 10% of the thickness of a corresponding barrier film comprising 38 mol % EVOH.

The present subject matter also includes the use of one or more metal oxide layers as barrier layer(s). For example, a metal oxide layer applied by vapor deposition may in certain applications be useful. A particular example is a thin layer of aluminum oxide vapor deposited on a polymeric substrate. It is also contemplated that one or more metal oxide layers and particularly aluminum oxide layers could be disposed, i.e. “sandwiched,” between two polymeric layers.

Thus, it will be understood that the present subject matter includes a barrier assembly with one or more barrier layers that comprise (i) EVOH, (ii) G-EVOH, (iii) elastomeric norbornenes, (iv) polyglycolic acid (PGA) resins or materials, (v) metal oxides, and (vi) combinations of (i)-(v). In certain versions of the present subject matter, the PGA resins are polyester polyglycolic acid resins. And, in certain versions of the present subject matter, the metal oxides are aluminum oxides.

### Other Functional Layer(s)

In addition to the previously noted films, the preferred embodiment multilayer barrier assemblies may also comprise additional functional layers. Preferably, the functional layers are all free of halogens. Non-limiting examples of suitable functional layers include outer protective layers, support layers, supplemental or secondary barrier layers, antimicrobial layers, and inner layers.

In certain embodiments, it may be preferred to utilize a PVOH-based barrier coating in addition to the barrier layers described herein or instead of the barrier layers described herein. An example of a suitable PVOH-based barrier coating and which is commercially available is NANOSEAL Barrier Coating NSC-100A available from NanoPack, Inc. of Wayne, Pa. It is also believed that a coating material available from NanoPack under the designation NSC-70 would also be suitable for certain applications.

NanoSeal coatings are aqueous dispersions of vermiculite platelets in polyvinyl alcohol (PVOH). The platelets are 1-3 nanometers thick and 10-30 microns in breadth, yielding an average aspect ratio (breadth-to-thickness) of approximately 10,000:1. The platelets are maintained in singularized format when dispersed into the PVOH resin and aligned in the plane of the coating when deposited on a film substrate, such as polyethylene terephthalate (PET). The dispersed platelets create a tortuous path for gas molecules, enabling extraordinarily high gas barrier in a very thin layer (0.2-0.5 microns dry) with excellent bond strength, clarity and flexibility. The proprietary formulation also improves the moisture-resistance of the PVOH resin.

The key technical difference between NanoSeal coatings and past attempts to use nanoclays for barrier enhancement is that of NanoSeal coatings the clay platelets remain in singularized format not only when dispersed in the resin, but also during and after deposition. This achievement presents the tortuous path required for high gas barrier, while enabling the cohesive strength necessary for multi-layered packaging film structures.

Although not wishing to be bound to any particular type of clay, for many applications nano particles of vermiculite are preferred.

The thickness of such a barrier coating can in many instances depend upon the requirements of the particular application. However, a coating thickness of from about 0.6 to about 0.1 microns and preferably about 0.3 microns is preferred.

It is also contemplated that a nanoclay barrier coating can be applied on a layer including about 10% elastomeric COC and about 90% EVA or one or more other polar materials. The elastomeric COC protects the barrier coating. The nanoclay component which is preferably vermiculite, also serves to provide an effect odor barrier.

As stated previously, in addition to barrier properties, it is often desirable that a polymeric barrier film not emit noise when deflected, crumpled or otherwise moved. For example, in ostomy or incontinence applications, it is desirable that the ostomy or incontinence bag not emit noise. As will be appreciated, such articles are typically worn under a user's clothing so as to hide the article from view. Films or polymeric layers that are not quiet tend to emit undesirable noise when the user undergoes motion such as when walking or sitting. In the case of the preferred embodiment multilayer barrier films, the films are significantly quieter than comparable ostomy films.

The preferred embodiment barrier film construction is believed to exhibit several advantages over currently known ostomy films. The preferred films are halogen-free and avoid the use of polyvinylidene chloride (PVDC). The preferred films are quieter and exhibit significantly less “rustle”. And, the preferred films exhibit superior odor blocking characteristics. Furthermore, the preferred films exhibit a combination of some and preferably all of these features. The film construction may be transparent or contain coloring agents.

Many other benefits will no doubt become apparent from future application and development of this technology.

All patents, published applications, and articles noted herein are hereby incorporated by reference in their entirety.

It will be understood that any one or more feature or component of one embodiment described herein can be combined with one or more other features or components of another embodiment. Thus, the present subject matter includes any and all combinations of components or features of the embodiments described herein.

As described hereinabove, the present subject matter solves many problems associated with previously known barrier films. However, it will be appreciated that various changes in the details, materials and arrangements of layers and materials, which have been herein described and illustrated in order to explain the nature of the subject matter, may be made by those skilled in the art without departing from the principle and scope of the subject matter, as expressed in the appended claims.

1. A multilayer barrier assembly comprising:
a first layer including at least one of EVOH and G-EVOH; and
a second layer including one or more elastomeric norbornenes,
wherein the first and second layers when combined exhibit a 2% secant modulus of less than 30 kpsi in both a machine direction (MD) and a cross direction (CD).

2. The multilayer barrier assembly of claim 1 wherein the first layer includes EVOH.

3. The multilayer barrier assembly of claim 1 wherein the first layer includes G-EVOH.

4. The multilayer barrier assembly of claim 1 wherein the second layer also includes a tie component.

5. The multilayer barrier assembly of claim 4 wherein the tie component is GMAH-EVA.

6. The multilayer barrier assembly of claim 1 further comprising:
an outer layer.

7. The multilayer barrier assembly of claim 1 further comprising:
at least one flexible support layer.

8. The multilayer barrier assembly of claim 7 wherein the at least one flexible support layer includes a first flexible support layer and a second flexible support layer.

9. The multilayer barrier assembly of claim 1 wherein the elastomeric norbornene is an elastomeric COC.

10. The multilayer barrier assembly of claim 1 wherein the first layer also includes at least one of a PGA material and a metallic oxide.

11. A multilayer barrier assembly comprising:
at least one layer including G-EVOH.

12. The multilayer barrier assembly of claim 11 further comprising:
an outer layer; and
an inner layer;
wherein the at least one layer including G-EVOH is disposed between the outer layer and the inner layer.

13. The multilayer barrier assembly of claim 11 further comprising:
a first flexible support.

14. The multilayer barrier assembly of claim 11 further comprising:
a first tie layer including a tie component.

15. The multilayer barrier assembly of claim 14 further comprising:
a second tie layer including a tie component.

16. The multilayer barrier assembly of claim 13 further comprising:
a second flexible support.

17. The multilayer barrier assembly of claim 11 further comprising:
at least one layer including one or more elastomeric norbornenes.

18. The multilayer barrier assembly of claim 17 wherein the elastomeric norbornenes are elastomeric COC's.

19. The multilayer barrier assembly of claim 14 wherein the tie component is GMAH-EVA.

20. The multilayer barrier assembly of claim 15 wherein at least one of the first tie layer and the second tie layer includes an elastomeric norbornene.

21. The multilayer barrier assembly of claim 11 wherein the layer including G-EVOH also includes at least one of a PGA material and a metallic oxide.

22. A multilayer barrier assembly comprising:
an outer layer;
a first flexible support;
a first tie layer including a tie component;
a barrier layer including at least one of EVOH, G-EVOH, PGA material, and metal oxide(s);
a second tie layer including a tie component;
a second flexible support; and
an inner layer.

23. The multilayer barrier assembly of claim 22 wherein the barrier layer includes EVOH.

24. The multilayer barrier assembly of claim 22 wherein the barrier layer includes G-EVOH.

25. The multilayer barrier assembly of claim 22 wherein at least one of the first tie layer and the second tie layer also includes one or more elastomeric norbornenes.

26. The multilayer barrier assembly of claim 25 wherein both the first tie layer and the second tie layer include one or more elastomeric norbornenes.

27. The multilayer barrier of claim 25 wherein the elastomeric norbornene is an elastomeric COC.

28. The multilayer barrier of claim 22 wherein the tie component is GMAH-EVA.

29. An ostomy barrier assembly comprising:
an outer layer;
a first flexible support;
a first tie layer including a tie component;
a barrier layer including at least one of EVOH, G-EVOH, PGA material, and metal oxide(s);
a second tie layer including a tie component;
a second flexible support; and
an inner layer.

30. The ostomy barrier assembly of claim 29 wherein the barrier layer includes EVOH.

31. The ostomy barrier assembly of claim 29 wherein the barrier layer includes G-EVOH.

32. The ostomy barrier assembly of claim 29 wherein at least one of the first tie layer and the second tie layer also includes one or more elastomeric norbornenes.

33. The ostomy barrier assembly of claim 32 wherein both the first tie layer and the second tie layer include one or more elastomeric norbornenes.

34. The ostomy barrier of claim 32 wherein the elastomeric norbornene is an elastomeric COC.

35. A multilayer barrier assembly comprising:
an outer layer;
a first flexible support;
a second flexible support;
a first general barrier layer;
a barrier layer including EVOH;
a second general barrier layer;
a third flexible support;
a functional layer; and
an inner layer.

36. The multilayer barrier assembly of claim 35 wherein at least one of the first general barrier layer and the second general barrier layer includes one or more elastomeric norbornenes.

37. The multilayer barrier assembly of claim 36 wherein the elastomeric norbornene is an elastomeric COC.

38. The multilayer barrier assembly of claim 35 wherein the barrier layer further includes at least one agent selected from the group consisting of G-EVOH, elastomeric norbornenes, PGA material, metal oxide(s), and combinations thereof.
